(19) United States
(12) Patent Application Publication
(10) Pub. No.: US 2006/0121097 A1
(43) Pub. Date: Jun. 8, 2006

(54) Treatment articles capable of conforming to an underlying shape

(76) Inventors: Richard Worthington Lodge, Cincinnati, OH (US); Miwa Miyamoto, Kobe (JP); Kouichi Miyamoto, Hyogo (JP); Kazuyuki Ohnishi, Takaishi (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/270,949

(22) Filed: Nov. 12, 2005

Related U.S. Application Data
(60) Provisional application No. 60/627,566, filed on Nov. 12, 2004.

(51) Int. Cl. A61K 9/70

(52) U.S. Cl. 424/443

(57) ABSTRACT

Treatment articles capable of conforming to an underlying shape and methods for their use are provided. The articles comprise a water insoluble substrate having at least one elongation zone where at least a portion of the elongation zone has been incrementally stretched. The article also includes a treatment composition which preferably delivers a skin care active. Preferred articles of the present invention comprise facial treatment masks.

### CROSS-REFERENCE TO RELATED APPLICATION

This application claims the benefit of U.S. Provisional Application No. 60/627,566, filed on Nov. 12, 2004.

### FIELD OF THE INVENTION

The present invention relates to treatment articles which are capable of conforming to an underlying shape such as the human body and particularly to masks capable of conforming to the contours of the face.

### BACKGROUND OF THE INVENTION

Cosmetic treatment articles such as wipes and masks are widely known in the art. These treatment articles commonly comprise a porous, water insoluble substrate having a cosmetic treatment composition loaded onto the substrate. The articles are then applied to mammalian skin where the treatment composition is transferred to the skin. These cosmetic articles are commonly applied to the skin for cleansing and the overall care of the skin as well as to improve the health and physical appearance of the skin. Rough and/or broken skin, wrinkling, excessive oil and hyperpigmentations (such as age spots, freckles and discoloration associated with sunlight, skin aging and environmental damage) are skin conditions commonly treated.

Cosmetic treatment articles typically comprise flat two dimensional substrate materials which are incapable of fitting to three dimensional surfaces containing double curvature. Unfortunately, skin surfaces to which these substrates are applied, frequently are three dimensional double curvature surfaces, for example the nose, forehead and/or chin of the face. These flat two dimensional substrates have a tendency to form a gap between the skin surface and the article and as a result do not fit to these curved surfaces well. The treatment composition loaded onto the substrate will then fail to properly contact the skin.

One solution to the aforementioned problem has been the use of slits in selective locations on the substrate. Slits or cuts provide the ability for the substrate to be overlayed or folded upon itself in the region of the slit or cut providing the article the ability to contact more area of the skin in the aforementioned three dimensional double curvature areas. While the use of such slits or cuts has achieved widespread commercial acceptance and commercial success, these articles continue to display a tendency to gap and therefore deliver less than the optimal treatment composition to the skin.

A cosmetic article that readily conforms to the double curved surfaces of the skin offers many advantages. The article will deliver an optimal amount of the treatment composition to the surface of the skin as there is contact between the substrate and the skin at an increased number of points along the substrate surface. In addition, there is increased comfort to the wearer of the article. Such cosmetic articles are only weakly adhered to the skin surface. Increased contact between the article and the skin will increase the adhesion and provide greater comfort.

Accordingly, the need remains for a treatment article which readily conforms to curved surfaces thereby delivering the aforementioned advantages of increased effectiveness and comfort.

### SUMMARY OF THE INVENTION

This need is met by the present invention wherein a treatment article having a water insoluble substrate and a treatment composition is provided. The substrate has at least one elongation zone in which incremental stretch has been provided. Preferably, the elongation zone comprises at least a first region and a second region. The first region is an area or areas within the substrate in which the substrate has been incrementally stretched so as to increase its surface pathlength in relation to a surface pathlength in a second region. This incremental stretching provides the article substrate with a zone or area within the substrate which is capable of more properly conforming to the contours of double curved surfaces. Thus, articles such as wipes and masks may be formed which readily conform to the face or other areas of the body which reduce or even eliminate the use of slits or cuts in the substrate surface providing for improved surface contact between the face or body and the cosmetic treatment article.

In addition, the articles provide greater comfort and flexibility to the wearer. While not wishing to be bound by theory, it is believed that the articles of the present invention provide better adherence to the skin of the body due to the increased surface contact. This improved adherence provides the wearer of the article with greater range of motion allowing more flexibility than traditional cosmetic articles such as masks which require consumers to remain essentially immobile to prevent the mask from pulling away from the skin.

According to a first embodiment of the present invention, a treatment article is provided. The article comprises a water insoluble substrate having at least one elongation zone wherein at least a portion of the elongation zone has been provided with incremental stretch. Preferably, the elongation zone comprises at least a first region and at least a second region. The first region has been provided with incremental stretch giving the first region a surface pathlength which is measured generally parallel to a defined direction and which is different than a second surface pathlength within the second region. In addition, the article has a treatment composition, preferably incorporated onto the substrate. The treatment article may take the form of most any cosmetic article for treatment of the skin, but is in preferred embodiments a facial treatment or cleansing mask.

In accordance with a second embodiment of the present invention, a cosmetic treatment method is provided. The cosmetic treatment method involves the steps of providing an article having at least one elongation zone wherein at least a portion of the elongation zone has been provided with incremental stretch, applying the article to a region of the body, conforming the elongation zone to the contours of the body and allowing the article to contact the body for a period of time effective to provide cosmetic treatment. The elongation zone of the article preferably comprises a first region which has been provided with incremental stretch giving the first region a surface pathlength which is measured generally parallel to a defined direction and where the first surface pathlength is greater than a second surface pathlength in the second region. In a preferred method, the article is a facial treatment or cleansing mask and the region of the body to which it is applied is the facial region.

The objects and features of the present invention will be more readily apparent from the following description when considered in conjunction with the accompanying drawing and appended claims.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings.

FIG. 1 is a planar view of a preferred embodiment of the article of the present invention.

FIG. 2 is a perspective view of a portion of one embodiment of the present invention.

FIG. 3 is an inflated cross sectional view of a preferred embodiment of the present invention with the article having an occluded side

FIG. 4 is a planar view of a preferred embodiment of the article of the present invention.

FIG. 5 is an illustration of an incremental stretching system according to the present invention.

### DETAILED DESCRIPTION OF THE INVENTION

According to the present invention, articles which provide superior fit to the contours of the skin are provided. In particular, the articles of the present invention more readily conform to the curvature of the human body such as the face. This improved fit of the articles, and in particular facial masks, provide improved surface contact between the face or body and the treatment article. In addition, the articles provide greater comfort and flexibility to the wearer and provides better adherence to the skin of the body and greater range of motion of the wearer.

While the article of the present invention will be described in the context of a facial mask, one of ordinary skill in the art will recognize the ability to and be able to readily adapt the present invention to treatment articles in general. As used herein, the term “treatment article” and “cosmetic treatment article” refers to devices which are adapted for application to the body, and in particular, to the human body. More specifically, “cosmetic articles” are devices for personal care or cosmetic devices, including wipes, facial masks and the like.

In addition, “comprising” means that other steps and other ingredients which do not affect the end result can be added. This term encompasses the terms “consisting of” and “consisting essentially of”.

The phrase “topical application” means to apply or spread a material onto the surface of the skin.

The phrase “cosmetically-acceptable” means that the compositions or components thereof so described are suitable for use in contact with human skin without undue toxicity, incompatibility, instability, allergic response, and the like.

The phase “mixtures” is meant to include a simple combination of materials and any compounds that may result from their combination.

All ingredients such as actives and other ingredients useful herein may be categorized or described by their cosmetic and/or therapeutic benefit or their postulated mode of action. However, it is to be understood that the actives and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

All percentages, parts and ratios are based upon the total weight of the compositions of the present invention, unless otherwise specified. All such weights as they pertain to listed ingredients are based on the active level and, therefore do not include carriers or by-products that may be included in commercially available materials.

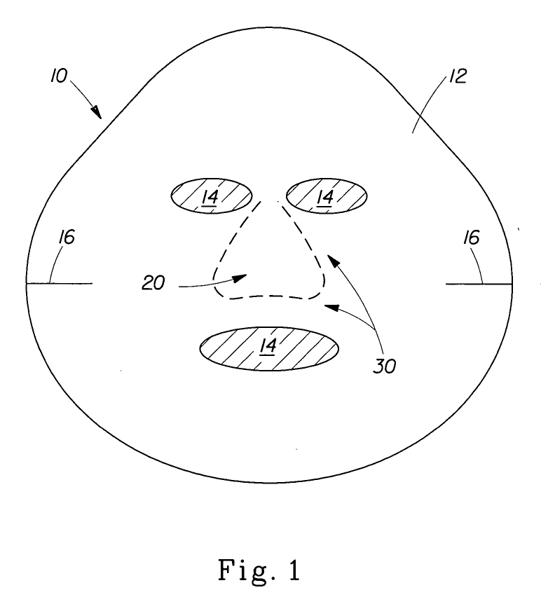

A preferred embodiment of the present invention comprises a cosmetic article such as a treatment mask for application to the human body. Turning to FIG. 1, an article 10 having the general shape of the human face is provided. Article 10 includes a water insoluble substrate 12, openings 14, a first region 20 and a second region 30. The article 10 of FIG. 1 is depicted as a whole, single piece facial mask. However, it will be readily apparent to one of ordinary skill that the articles of the present invention may comprise a single piece or a multi-part article. For example, a multi-part mask is configured to cover substantially the whole area of the facial skin, and is made of multiple pieces. Most commonly, multi-part masks comprise two-part masks having a first piece covering the upper area of the face, i.e. the nose and there above, and a second piece covering the lower area of the face, i.e. the upper lip, cheeks and there under. Of course, one of ordinary skill will recognize that articles having three or more pieces may be envisioned. In addition, the exact areas of coverage of multi-part masks as to each individual piece may vary. Single piece facial masks may cover substantial the whole face or be configured to match the area of a particular part of the face, such as the nose, cheekbone, chin, forehead, or combinations thereof. In another preferred embodiment, the mask is so configured to have ears, pulls, or rings for facilitating placement and/or removal of the mask on the skin.

When configured to the areas surrounding the eyes or nostrils, the article 10 may be so configured to cover substantially the whole area of the facial skin in these general areas with areas of the eyes and nostrils opened. Thus, referring again to FIG. 1, mask 10 includes substrate 12. The outer peripheral of substrate 12 is designed to approximately match the outline of the face, with a plurality of openings 14 for the eyes and the mouth. For single and multi-part masks designed to cover the whole face, the substrate 12 preferably has a length of from about 15 cm to about 25 cm, more preferably from about 18 cm to about 23 cm, and a preferably width of from about 15 cm to about 30 cm, more preferably from about 20 cm to about 25 cm; to cover the average entire facial area. Of course, multi-part masks may be formed with pieces of varying size and with areas of overlap. In such instances, the areas of the multi-part mask in contact with the skin preferably conform to the above stated dimensions. A plurality of slits, cuts or line of weakness 16 may be provided in the article of the present invention.

Substrate 12 of the present invention comprises a water-insoluble material. By “water insoluble”, it is meant that the substrate does not dissolve in or readily break apart upon immersion in water. The water-insoluble substrate is the implement or vehicle for delivering the treatment composition.

A wide variety of materials can be used as the substrate. The following nonlimiting characteristics are desirable: (i) sufficient wet strength for use, (ii) sufficient softness, (iii) sufficient thickness, (iv) appropriate size, (v) air permeability, (vi) hydrophilicity and (vii) its ability to take permanent incremental strain, that is, its ability to be incrementally stretched per the following description.

Substrate 12 may be comprised of a variety of materials both natural and synthetic. By “natural”, it is meant that the materials are derived from plants, animals, insects or byproducts of plants, animals, and insects. By “synthetic”, it is meant that the materials are obtained primarily from various man-made materials or from natural materials which have been further altered. The conventional base starting material is usually a fibrous web comprising any of the common synthetic or natural textile-length fibers, or mixtures thereof.

Suitable examples of natural materials useful in the present invention are silk fibers, keratin fibers and cellulosic fibers. Keratin fibers may include those selected from the group consisting of wool fibers, camel hair fibers, and the like. Nonlimiting examples of cellulosic fibers include those selected from the group consisting of wood pulp fibers, cotton fibers, hemp fibers, jute fibers, flax fibers, and mixtures thereof.

Suitable examples of synthetic materials useful in the present invention include those selected from the group consisting of acetate fibers, acrylic fibers, cellulose ester fibers, modacrylic fibers, polyamide fibers, polyester fibers, polyolefin fibers, polyvinyl alcohol fibers, rayon fibers, polyurethane foam, and mixtures thereof. Examples of some of these synthetic materials include acrylics such as acrilan, creslan, and the acrylonitrile-based fiber, orlon; cellulose ester fibers such as cellulose acetate, amel, and acele; polyamides such as nylons (e.g., nylon 6, nylon 66, nylon 610, and the like); polyesters such as fortrel, kodel, and the polyethylene terephthalate fiber, dacron; polyolefins such as polypropylene, polyethylene; polyvinyl acetate fibers; polyurethane foams and mixtures thereof. These and other suitable fibers and the nonwoven materials prepared therefrom are generally described in Riedel, “Nonwoven Bonding Methods and Materials,” Nonwoven World (1987); The Encyclopedia Americana, vol. 11, pp. 147-153, and vol. 26, pp. 566-581 (1984); U.S. Pat. No. 4,891,227, to Thaman et al., issued Jan. 2, 1990; and U.S. Pat. No. 4,891,228 which are all incorporated by reference herein in their entirety.

Nonwoven substrates may be employed in the present invention. Nonwoven substrates may be formed from natural materials consisting of webs or sheets and are commonly formed on a fine wire screen from a liquid suspension of the fibers. See C. A. Hampel et al., The Encyclopedia of Chemistry, third edition, 1973, pp. 793-795 (1973); The Encyclopedia Americana, vol. 21, pp. 376-383 (1984); and G. A. Smook, Handbook of Pulp and Paper Technologies, Technical Association for the Pulp and Paper Industry (1986); which are incorporated by reference herein in their entirety.

The substrate can be made into a wide variety of shapes and forms such as flat pads, thick pads, thin sheets, and sheets of irregular thickness, depending on the desired use and characteristic of the article. The substrate is typically designed to fit the area of the skin to which topical application is desired. For example, when the article is applied to the face, the substrate is designed to correspond to the shape of the face avoiding the eye, nostril, and mouth areas, as necessary.

Substrate 12 is flexible enough such that, when impregnated with a treatment composition, it readily fits along the skin, yet is strong enough so that it does not easily tear or crumble upon use. Preferably, the substrate has a thickness of from about 100 μm to about 1 cm, more preferably from about 300 μm to about 3 mm, depending on the material for making the substrate, and use and characteristic of the product.

When the substrate consists of multiple layers, the substrates can include films, laminates and other non-fibrous materials. In one embodiment, the substrate may also be provided with a backing layer. The backing layer preferably is a polymer film such as polypropylene or polyethylene. The backing layer may be attached to the other layer or layers of substrate 12 via lamination, coating or heat sealing. The resulting substrate with the backing layer comprises an occluded side on one side of the substrate, which faces away from the skin, and a skin facing side that is positioned on the skin surface. By having a substrate with an occluded side, the substrate acquires low air permeability. By “low air permeability” it is meant that the side of the substrate having the backing layer reduces the amount of air entering into the substrate and reduces the amount of vapor escaping from the substrate. Preferably the air permeability of the backing layer is less than about 5 mg/cm²/min, more preferably between about 0.01 mg/cm²/min and about 4.8 mg/cm²/min. The air permeability can be measured by taking the weight of a fully saturated sample of the substrate and weighing the substrate after it is exposed to the atmosphere.

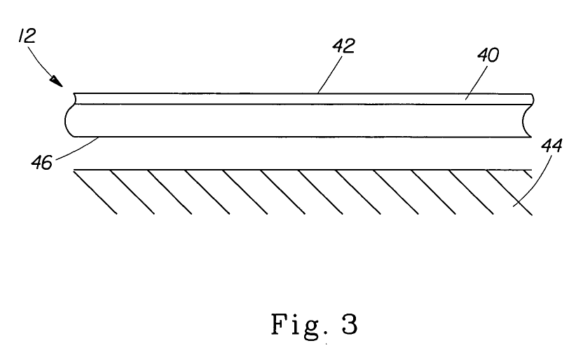

A preferred embodiment of the present invention having an occluded side having low air permeability is shown in FIG. 3. As shown in FIG. 3, a substrate 12 is laminated with a polymer film 40, forming an occluded side 42. The polymer film 40 is greatly exaggerated to show detail. The occluded side 42 is placed away from the skin surface 44 during use. The occluded side 42 restricts moisture from escaping into the atmosphere during a typical use time period between about 5 minutes to about 45 minutes thus providing improved moisturizing effect to the skin. The skin facing side 46 of the substrate 12 preferably does not contain any materials that restrict air permeability.

A substrate comprising an occluded side significantly increases penetration of certain skin care agents into the skin compared to a substrate without an occluded side. Without being limited by a theory, it is believed that the occluded side of the substrate allows for the creation of a humid environment near the surface of the skin by limiting the evaporation of water from the mask composition into the atmosphere. Additionally, the articles of the present invention preferably utilize water tension to adhere to the skin surface rather than strong adhesives on the skin facing side. The absence of a strong adhesive between the substrate and the skin surface, as utilized by preferred embodiments of the present invention, removes the physical barrier resulting from the strong adhesive and promotes the penetration of skin care agents. The resulting environment between the skin and the substrate promotes the penetration of the skin care agents into the skin.

Suitable polymeric materials for the backing layer include polyethylene, polypropylene, polyethylene terephtalate, polyamides, polyesters, nylons, blends thereof, or any other cosmetically acceptable polymeric films. Suitable coatings include any materials know in the art that impart low air permeability to the substrate and are cosmetically acceptable. Heat-sealing the substrate may be accomplished by any method known in the art to impart low air permeability to the substrate.

Substrate materials particularly preferred herein include those that are hydrophilic in nature, thereby capable of absorbing a larger quantity of the composition. In these preferred embodiments, the water-insoluble substrate can be made solely of hydrophilic material, or made of layers of substrates wherein at least the layer facing the skin is made of hydrophilic material.

In alternative preferred substrates, the substrate may also be made by a combination of hydrophilic material and hydrophobic material. In one preferred embodiment, the substrate is made at least partially of hydrophilic materials selected from cotton, pulp, rayon, and mixtures thereof. By partially, it is meant to encompass both situations where at least a layer of a hydrophilic substrate is used, and where at least some of the hydrophilic material is used in combination with another material to make a mixed substrate. In one embodiment the mixed substrate comprises materials having increasing hydrophobic properties from the skin facing side 46 to the occluded side 42. Referring to FIG. 3, the skin facing side 46 of the substrate faces the skin and comprises hydrophilic material. Moving through the cross section of the substrate 12 to the occluded side 42 that is in contact with the ambient air, the substrate utilizes more hydrophobic materials. By “hydrophobic” it is meant that the materials in this portion of the substrate have a contact angle with moisture greater than about 10 degrees, preferably between about 10 degrees and 90 degrees as measured by ASTM D 5725-99. This structure allows for moisture to be move from the hydrophobic occluded side of the substrate to the hydrophilic skin facing side of the substrate to the skin surface. This preferred structure could be accomplished by having a single layer or a laminated structure.

Substrates made from natural materials useful in the present invention can be obtained from a wide variety of commercial sources. Nonlimiting examples of suitable commercially available paper layers useful herein include AIRTEX®, an embossed airlaid cellulosic layer having a base weight of about 71 gsy (grams per square yard), i.e., about 85 gsm (grams per square meter), available from James River, Green Bay, Wis.; and WALKISOFT®, an embossed airlaid cellulosic having a base weight of about 75 gsy (about 90 gsm), available from Walkisoft U.S.A., Mount Holly, N.C.

Nonwoven substrates made from synthetic materials useful in the present invention can also be obtained from a wide variety of commercial sources. Nonlimiting examples of suitable nonwoven layer materials useful herein include NOVONET® 149-616, a thermo-bonded grid patterned material containing about 100% polypropylene, and having a basis weight of about 50 gsy (about 60 gsm), available from Veratec, Inc., Walpole, Mass.; NOVONET® 149-801, a thermo-bonded grid patterned material containing about 69% rayon, about 25% polypropylene, and about 6% cotton, and having a basis weight of about 75 gsy (about 90 gsm), available from Veratec, Inc. Walpole, Mass.; NOVONET® 149-191, a thermo-bonded grid patterned material containing about 69% rayon, about 25% polypropylene, and about 6% cotton, and having a basis weight of about 100 gsy (about 120 gsm), available from Veratec, Inc. Walpole, Mass.; KEYBAK® 951V, a dry formed apertured material, containing about 75% rayon, about 25% acrylic fibers, and having a basis weight of about 43 gsy (about 51 gsm), available from PGI/Chicopee, Dayton, N.J.; KEYBAK® 1368, an apertured material, containing about 75% rayon, about 25% polyester, and having a basis weight of about 39 gsy (about 47 gsm), available from PGI/Chicopee, Dayton, N.J., RMT-90, a 3-layer substrate having a pulp layer as an inner layer with outer layers respectively made of the combination of rayon and polyester, and RFP-90, a 3-layer substrate having a PP carded and thermal bonded layer as an inner layer with outer layers of rayon, both available from Daiwabo K. K. Daiwabo DFS(SH)T-70 is a 3-layer substrate (carded and hydroentangled nonwoven) having a pulp layer as an inner layer with outer layers of PE/PET splitting fiber, available from Daiwabo K. K. of Japan. The total basis weight of Daiwabo DFS(SH)T-70 is about 70 gsm (pulp; about 15 gsm, PE/PET splitting fiber; about 55 gsm). Further suitable substrates include Kuraray TT 463Q60, a single-layer substrate (carded and hydroentangled nonwoven) having a layer of PET fiber. The PET resin is chemically treated to be more hydrophilic. The total basis weight is about 60 gsm.

In the present invention the nonwoven layer can be prepared by a variety of processes including hydroentanglement, thermally bonding or thermo-bonding, and combinations of these processes. Methods of making nonwoven substrates are well known in the art. Generally, these nonwoven substrates can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed.

Processes for preparing hydroentangled webs are well known in the art. See, for example, Evans; U.S. Pat. No. 3,485,786; issued Dec. 23, 1969; Kalwarres; U.S. Pat. No. 2,862,251 and Griswold; U.S. Pat. No. 3,025,585, all of which describe hydroentangling procedures generally and all of which are herein incorporated by reference. See also U.S. Pat. No. 5,674,591; James et al; issued Oct. 7, 1997 which specifically describes a hydroentangling process, including the apparatus used in said process, which can be used to prepare the patterned web. U.S. Pat. No. 5,674,591 is incorporated herein in its entirety.

Among the water-insoluble substrate described hereinbefore, fluid-entangled (hydroentangled), nonwoven, flexible substrates are preferably used in the present invention, in view of providing softness, loftness, strength while being used, and cloth-like texture. Any fluid-entangled, nonwoven, flexible substrate that is known or is otherwise suitable for application to the skin can be used in the mask compositions of the present invention.

The term “fluid-entangled” as used herein is an art recognized term which refers generally to the manufacturing process for entangling a fibrous web by using a fluid jet on a fibrous web to obtain the desired fiber and void configuration within the resulting fluid-entangled substrate, to thereby produce an art recognized, fluid-entangled, nonwoven, flexible substrate. Fluid-entangled, nonwoven, flexible substrates and the fluid entangling techniques for making them are well known in the substrate arts, preferred examples of such substrates and fluid entangling techniques being described in U.S. Pat. No. 5,142,752 (Greenway et al.) and U.S. Pat. No. 5,281,461 (Greenway et al.), which patents are incorporated herein by reference in their entirety.

These substrates are preferably a blend of polyester and/or polyolefin and natural fiber such as pulp, rayon or cotton fibers and have a weight in the range of 40-120 gsy (grams per square yard), i.e., 48-144 gsm (grams per square meter). If synthetic fibers such as polyester or polyolefin are used, preferably they are fine fibers to provide softness to the skin. Herein, “fine” means that the fibers are less than 1 dtex (1 dtex=1 g/10000 m). These preferred substrates are typically prepared by using an apparatus and related process for entangling a fibrous web which employs columnar fluid jets to eject a continuous curtain of fluid in an entangling station. The web is advanced through an entangling station on a conveyor which supports an entangling member having a symmetrical pattern of void areas. Baffle members disposed in the void areas are provided which include radiused curvatures and define apertures having a frusto-conical configuration. Dynamic forces in the fluid curtain impact the web in discrete and controlled patterns determined by the baffling members to enhance efficient energy transfer and web entanglement. Textile-like fabrics having a uniform, non-apertured, surface cover are obtained by coaction of fluid curtain and baffle structures.

Other known techniques for making fluid-entangled, nonwoven, flexible substrates are described, for example in U.S. Pat. No. 3,485,786 (Evans); U.S. Pat. No. 2,862,251 (Kalwarres); and U.S. Pat. No. 3,025,585 (Griswald), which descriptions are incorporated herein by reference.

Other suitable methods of making fluid-entangled substrates are described in U.S. Pat. No. 5,674,591 (James et al.) which specifically describes a hydroentangling process, including the apparatus used in said process, which can be used to prepare a patterned web, which description is incorporated herein by reference. The resulting layer, regardless of its method of production or composition, is then subjected to at least one of several types of bonding operations to anchor the individual fibers together to form a self-sustaining web.

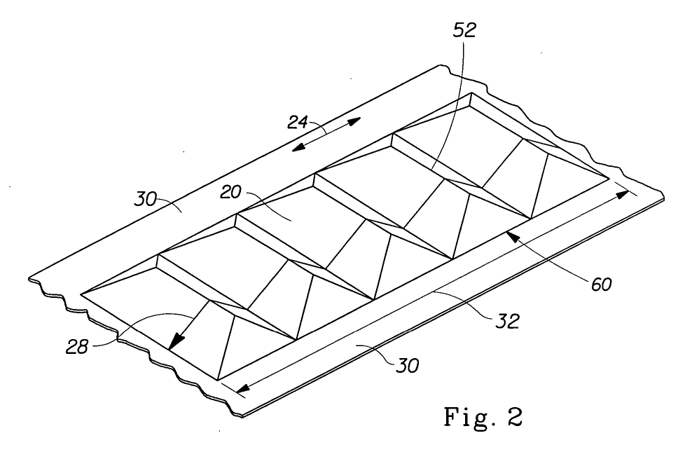

Referring to FIGS. 1-2, article 10 is shown in a relatively planar configuration. A slight force may be applied to the article 10 such that the substrate generally occupies a single plane. As used herein, the term “relatively planar” refers to objects having at least one surface that lies substantially within a single plane. At least a portion of substrate 12 comprises at least a first region 20 and a second region 30. First and second regions are preferably interconnected or interrelated regions. defining an elongation zone within the article which allow substrate 12 to extend or conform to a surface in response to an applied force. Substrate 12 includes a transition region 60 which is at the interface of first region 20 and second region 30. Transitional region 60 will generally exhibit combinations of the behavior of both the first and second region. However, the present invention is largely defined by the behavior of the substrate 12 in the distinctive regions, first region 20 and second region 30. Therefore, the ensuing description of the present invention will be concerned with the first region 20 and second region 30 as the performance of the article 10 is not dependent upon the transition region 60.

Regions within the present invention are those areas on substrate 12 in which the surface of the substrate is generally uniform. That is, in regions which are unaltered, the substrate is generally unaltered. In regions in which incremental stretching has been applied are defined as that area where the incremental stretching is generally uniform. For simple geometries of standard application of strain in one direction, such a region will be clear. For more complex geometries in which the direction or degree of strain is changing then the region refers to a smaller portion of the geometry in which the change in direction or strain is insignificant.

Substrate 12 in the area of first region 20 is formed with incremental stretching. As used herein, the terms “incremental stretching” or “incrementally stretched” refer to intermittently mechanically elongated regions of a substrate having a multiplicity of smaller increments which have been individually stretched beyond the plastic yield point of the material to effect permanent elongation. As used herein the term “formed” refers to the creation of a desired structure or geometry upon a substrate material that will substantially retain the desired structure or geometry when the substrate is not subjected to any externally applied elongation or forces.

The incremental stretching in first region 20 means the substrate 12 within the region will take the shape of raised ribs or ridges 52 formed in substrate 12. First region 20 may then expand via a general flattening of the ridges 52 in the general direction of the incremental stretching 24 when a force is applied. This allows the article 10 to conform to the underlying region of the body. The direction of incremental stretching 24 is the general direction in which strain has been applied to the substrate 12 and is typically a direction generally perpendicular to ridges 52. Such direction may include the machine direction, cross machine direction or some combination of the two.

When substrate 12 is subjected to an applied force, the substrate exhibits elastic like behavior as it extends generally in the direction of the applied force. Preferably, this applied force is generally perpendicular to the plane of article 10. In certain embodiments, the substrate 12 will return to its substantially untensioned condition once the applied force has been removed, provided the material is not extended beyond the point of yielding. Preferred substrate materials are capable of undergoing multiple cycles of applied force without losing the ability to substantially recover provided the material is not extended beyond the yielding point.

Any suitable method for providing incrementally stretching in the substrate 12 may be employed. Some nonlimiting preferred methods for providing the incrementally stretched regions are described in detail in U.S. Pat. No. 5,143,679 entitled “Method For Sequentially Stretching Zero Strain Stretch Laminate Web To Impart Elasticity Thereto Without Rupturing The Web” issued to Weber et al. on Sep. 1, 1992, which is hereby incorporated by reference. (Although the Weber et al. patent referenced above is a method for sequentially stretching a laminate web, it is recognized that such methods may be used to stretch any suitable web, including single ply and multiple ply webs, films laminates etc.) Alternatively, the article 10 of the present invention may be provided with incremental stretching by methods such as those described in U.S. Pat. No. 5,518,801 entitled Web Materials Exhibiting Elastic-Like Behavior, which issued to Chappell, et, al. on May 21, 1996, and U.S. Pat. No. 5,968,029 entitled Web Materials Exhibiting Elastic-like Behavior” issued to Chappell et al on Oct. 19, 1999 which are also incorporated by reference herein.

The first region 20 has a first surface pathlength 28. As used herein, the term “surface pathlength” refers to a measurement along the topographic surface of the region or zone in question in a direction generally parallel to an axis. The term “generally parallel” refers to an orientation between axes, elements or a combination of axes and elements whereby the subtended angle formed by the axes or elements being compared is less than 45 degrees. The term “generally perpendicular” refers to an orientation between axes, elements or a combination of axes and elements whereby the subtended angle formed by the axes or elements being compared is greater than 45 degrees.

In addition to first region 20, article 10 of the present invention is provided with a second region 30. Second region 30 is also provided with a second surface pathlength 32. Second region 30 represents an area in which the second surface pathlength 32 is different from that of first surface pathlength 28 in at least one defined direction. The second region 30 may comprise an unaltered substrate or be incrementally stretched to a different direction or degree than that in first region 20. Preferably, second region 30 will have a second surface pathlength 32 which is shorter than the first surface pathlength 28 of first region 20. This difference in pathlengths allows the article 10 to expand upon application to the body within at least the first region and allows article 10 to more readily fit the contours of the body. Most preferably, second surface pathlength is at least 3%, more preferably at least 10% and even more preferably at least 30% less than the first surface pathlength 28. In another preferred embodiment, second region 30 is an unaltered substrate.

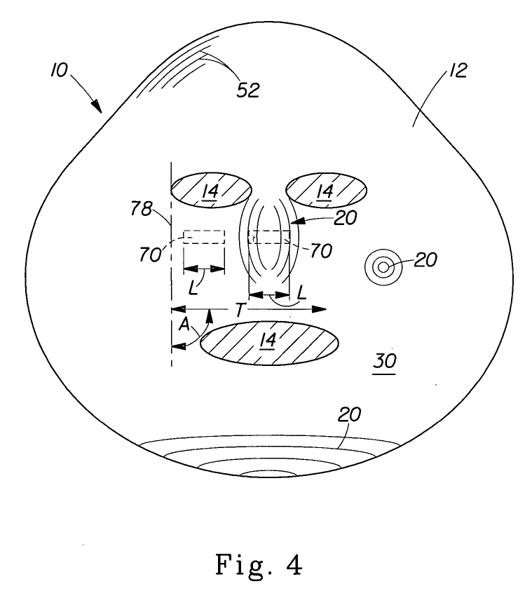

Further, while the article 10 may comprise a single first and second region, article 10 may also comprise a plurality of regions in various locations and geometries. Turning to now to FIG. 4, an article of the present invention having a multiplicity of first regions 20 and second regions 30 is shown.

“Surface pathlength” or “pathlength” is defined as a length of a substrate or any portion 70 thereof measured on the surface of article 10 generally parallel to a predetermined direction or the defined direction T. Preferably, the defined direction T is generally parallel to the direction of incremental stretching 24 as shown in FIG. 2. However, one of ordinary skill in the art will recognize that if the direction of incremental stretching is not readily apparent the defined direction may be chosen arbitrarily.

Returning to FIG. 4, surface pathlength is determined when the portion 70 of the article 10 is measured in a relatively planar configuration. The surface pathlength is equivalent to the straight line spatial distance between opposite ends of a fixed portion 70 of the first and second regions. For simple geometries such as that shown in FIG. 1 where the first region 20 is generally uniformly elongated in one direction, surface pathlength can be measured relatively easily. A portion 70 of the substrate within first region 20 of a defined length L is determined when substrate is in its original post-stretched state. That is, before a force has been applied to the substrate to expand the incrementally stretched portions. If a force has been applied to the substrate, the substrate must be returned to its original pre-expanded state. The defined length L can be any convenient length containing at least two ridges 52, but no longer than the length of first region 20.

Once the length L and direction T are determined, they may be reproduced to the same defined length L and defined direction T in the second portion of the substrate. Either the machine direction or the cross machine direction of the substrate may be employed as an axis 78 from which to measure the defined direction T so the identical defined direction T and defined length L is employed in the second region. That is, the angle A of the defined direction T from the machine direction may be measured and the same angle employed in the second region. Each portion is then cut out as narrow strips where the strip typically has a width no greater than half of the defined length L and removed from the substrate and the strips are then extended to be as flat as possible without inducing tension. The length of each piece is measured and then corresponds to the surface pathlength.

The strips are extended such that when the substrate is pulled flat, without generating any tension in the substrate 12, geometric folds, bends, wrinkles, or other “out of plane” structures are eliminated. If the substrate is very flexible, as is common for the substrate materials typically used, it can normally be stretched into a substantially flat condition without significant tension. However, it should be recognized by those skilled in the art that if the geometric folds, bends, wrinkles, or other “out of plane” structures are sufficiently stiff so as to require tension in the substrate to flatten them, surface pathlength must be measured by other known means. One example of measuring surface pathlength without pulling the substrate flat is described in U.S. Pat. No. 5,518,801 entitled Web Materials Exhibiting Elastic-Like Behavior, which issued to Chappell et, al. on May 21, 1996, which is hereby incorporated herein by reference.

If the direction of incremental stretching 24 within first portion 20 is formed in a variable direction, i.e. changing axis or curvilinear direction, the length selected for pathlength measurement should be selected such that the length is generally in one linear axis. Should selection of such a length be impractical, then a linear axis should be approximated which represents an average of the curvilinear direction of incremental stretching.

While the test method described above is useful for many of the substrates of the present invention it is recognized that the test method may have to be modified to accommodate some of the more complex substrate materials within the scope of the present invention.

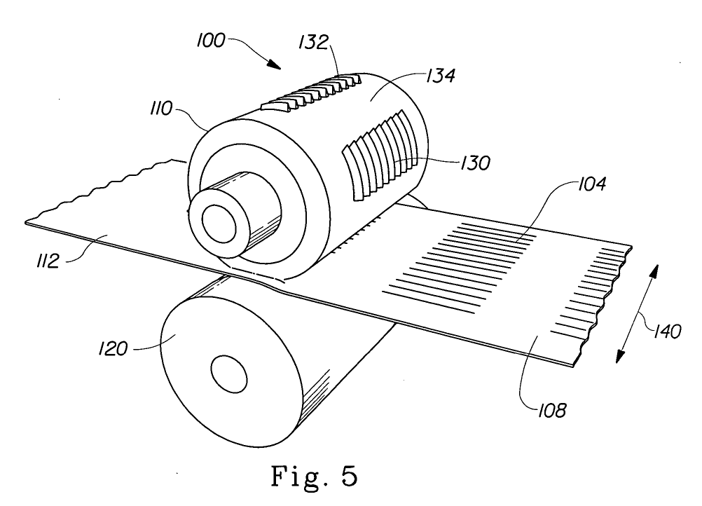

Preferred methods of forming the first portion 20 of the present invention include, but are not limited to embossing by mating plates or rolls, thermoforming, high pressure hydraulic forming, or casting. Preferably, an incremental stretching system is employed in elongating the first portion 20 of the present invention. Turning to FIG. 5, one preferred incremental stretching system 100 of the present invention is provided. The system 100 includes a pair of opposed intermeshing incremental stretching rollers consisting of a pair of opposed rollers defined as upper roller 110 and a lower roller 120. The rollers include a plurality of teeth 130 and corresponding grooves 132 which extend about the circumference of the rollers for varying distances depending upon the pattern of incremental stretching. Thus, teeth 130 on upper roller 110 intermesh with groove 132 on opposed lower roller 120, while teeth 130 on lower roller 120 intermesh with groove 132 on upper roller 110. Teeth 130 are interrupted on rollers 110 and 120 with zones of no teeth 134. As substrate 112 passes through the intermeshing teeth of the opposed rollers, the substrate is stretched and/or elongated by the intermeshing teeth forming first regions 104. Likewise, zones of no teeth 134 produce second portion 108 on substrate 112. The substrate may then be subjected to additional processing to form a completed article such as cutting operations to form a treatment mask.

Depending upon the orientation of the teeth 130 and grooves 134, the substrate may be incrementally stretched in various directions such as the cross-machine direction 140 or in the machine direction, not shown. The portion of the web passing between the smooth portions will be generally unstrained. The exact configuration, spacing, dimensions, and overlap of opposing teeth and grooves and rollers can be adjusted, as desired, to produce the desired amount of stretch, geometry and dimensions of the resultant regions. For example, the height and spacing of the teeth and grooves determine the amount of incremental stretch provided to first region 104. Higher teeth and corresponding deeper grooves, stretch the substrate to greater extent than shorter teeth and shallower grooves giving the substrate 112 a larger amount of stretch when applied to the body. Further, the shape of the teeth and corresponding grooves may vary depending on the desired configuration of the ridges on the substrate. Teeth having curved tops, flat tops or pointed tops all of varying dimensions are within the scope of this invention.

In addition, to the roller system described above, the first and second regions of the present invention may be formed via the use of mating plates. The resultant desired geometry of the first region is formed via the use of corresponding teeth and grooves on a pair of opposed plates. The plates are designed such that teeth on one plate intermesh with the grooves on the opposing plate. A portion of a web is then compressed between the plates to provide the incremental stretching to the first region. Again, the exact configuration, spacing, dimensions, and overlap of opposing teeth and grooves on the plates can be adjusted, as desired, to produce the desired amount of stretch, geometry and dimensions of the resultant first region.

One of ordinary skill in the art will recognize that as a plurality of first regions are provided in select embodiments, that differing regions may have different geometries and degrees of incremental stretching as dictated by the region of the body to which the article is applied.

The articles of the present invention include a treatment composition for application to the skin. The treatment composition may be provided on the substrate via a variety of means. The treatment composition may be provided in liquid form and soaked into the substrate. The substrate may than be packaged wet and sealed for delivery to consumers and immediate use upon opening of the sealed package. Alternatively, the treatment composition may be dried following application for re-wetting by the consumer prior to application. In yet another embodiment of the present invention, the treatment composition and the substrate may be packaged separately to be combined by the consumer prior to application to the skin. In such, instances the treatment composition may be provided either dry or in liquid form. While single use articles are the preferred embodiment of the present invention, one of ordinary skill in the art will recognize that a multi-use article is within the scope of the present invention.

The treatment composition of the present invention preferably includes a skin care active to provide a benefit to the skin. The skin care active of the present invention includes those as known in the art and includes anti-oxidants, cleansing agents, free radical scavengers, moisturizers, skin tone altering agents or depigmentation agents, anti-acne agents, anti-dandruff agents, anti-aging agents, softeners, anti-wrinkle agents, keratolic agents, anti-inflamatory agents, oily components, skin texture treatment agents, flavanoids, fresheners, healing agents, liporegulators, vascular protectors, anti-bacterials, agents, anti-fungal agents, anti-perspirant agents, deodorants, skin conditioners, anesthetics, nourishing agents, sebum absorbers, and moisture absorbers. The ingredients are described in more detail herein.

The treatments composition of the present invention may optionally include an oily component. Oily components useful herein can deliver skin conditioning benefits such as smoothness and softness to the skin. Oily components useful herein include, for example, fatty alcohols, silicone oils, mineral oil, petrolatum, C₇₋₄₀ straight and branched hydrocarbons such as isohexadecane, C₁₋₃₀ alcohol esters such as isopropyl isostearate, glycerides, alkylene glycol esters, propoxylated and ethoxylated derivatives, sugar ester such as sucrose polycottonseedate, vegetable oils such as coconut oil, hydrogenated vegetable oils, animal fats and oils, and C₄₋₂₀ alkyl ethers of polypropylene glycols, C₁₋₂₀ carboxylic acid esters of polypropylene glycols, and di-C₈₋₃₀ alkyl ethers. Hydrophobic nonionic surfactants, which are those being water-insoluble and having an HLB value of less than 10, can be used as oily components. Hydrophobic nonionic surfactants useful herein include, for example, cetearyl glucoside, steareth-2, laureth-4, sucrose cocate, sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

The composition of the present invention comprises hydrophilic surfactants. The hydrophilic surfactants are included in the composition at a level by weight of, preferably from about 0.01% to about 10%, more preferably from about 0.05% to about 5%, still more preferably from about 0.1% to about 2%.

Hydrophilic surfactants useful herein are those being water-soluble, and preferably have an HLB value of above 10. Hydrophilic surfactants useful herein include, for example, any cosmetically acceptable surfactants, i.e., nonionic surfactants, cationic surfactants, anionic surfactants, zwitterionic surfactants, amphoteric surfactants, and mixtures thereof. Among them, preferred herein are cosmetically acceptable nonionic surfactants in view of reduced skin irritation and conditioning benefits.

Hydrophilic nonionic surfactants useful herein include, for example, PEG- 100 stearate, polysorbate-20, polysorbate-60, seteareth-21, isoceteth-20, and oleth-20, laureth-23, ceteareth-12, steareth-100, PEG 40 hydrogenated castor oil, PEG-60 hydrogenated castor oil, and mixtures thereof.

The treatment composition may optionally include a skin texture treatment agent. Skin texture treatment agents help repair and replenish the natural moisture barrier function of the epidermis, thereby providing skin benefits such as texture improvement.

Skin texture improvement agents useful herein are niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, tocopheryl nicotinate, and vitamin B₃ and analogues or derivatives, and mixtures thereof. Panthenol is particularly preferred in that, when used in an amount of at least about 1%, it provides texture improvement benefits. Panthenol is commercially available, for example, by Roche. Skin vitalizing agents useful herein include seaweed extracts such as algae extract and Laminaria Digitata extract.

Anti-acne actives can be effective in treating and preventing acne vulgais, a chronic disorder of the pilosebaceous follicles. Preferred anti-acne agents optionally included in the treatment composition include salicylic acid, 4-methoxysalicylic acid, benzoyl peroxide, lactic acid, metronidazole, panthenol, retinoic acid and its derivaties, sulphur, triclosan, and mixtures thereof.

Anti-oxidants and radical scavengers are especially useful for providing protection against UV radiation which can cause increased scaling or texture changes in the stratum corneum and against other environmental agents which can cause skin damage and may be optionally included in the treatment composition.

Anti-oxidants and radical scavengers such as tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, amines (i.e., N,N-diethylhydroxylamine, amino-guanidine), sulfhydryl compounds (i.e., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts may be used. Preferred anti-oxidants/radical scavengers are selected from flavonoids and tocopherol sorbate and other esters of tocopherol, more preferably tocopherol sorbate. For example, the use of tocopherol sorbate in topical compositions and applicable to the present invention is described in U.S. Pat. No. 4,847,071, Bissett et al, issued Jul. 11, 1989.

Flavonoid compounds useful herein include unsubstituted flavanones, substituted flavanones, unsubstituted flavones, substituted flavones, unsubstituted chalcones, substituted chalcones, unsubstituted isoflavones, and substituted isoflavones. By the term “substituted” as used herein means flavonoid compounds wherein one or more hydrogen atoms of the skeleton structure as described above has been independently replaced with hydroxyl, C1-C8 alkyl, C1-C4 alkoxyl, O-glycoside, and the like or a mixture of these substituents. Flavonoid compounds particularly useful herein are selected from the group consisting of substituted flavanones, substituted flavones, substituted chalcones, substituted isoflavones, and mixtures thereof.

In one particularly preferred embodiment of the present invention, the glycoside flavonoid is selected from the group consisting of glucosyl hesperidin, glucosyl rutin, glucosyl myricitrin, glucosyl isoquercitrin, glucosyl quercitrin, methyl hesperidin, and mixtures thereof. These glucoside flavonoid compounds can be obtained by bio-chemical methods from related natural flavonoid compounds.

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

Suitable dipeptides for use herein include Carnosine® (beta-ala-his). Suitable tripeptides for use herein include, gly-his-lys, arg-lys-arg, his-gly-gly. Preferred tripeptides and derivatives thereof include palmitoyl-gly-his-1ys, which may be purchased as Biopeptide CL® (100 ppm of palmitoyl-gly-his-1ys commercially available from Sederma, France); Peptide CK (arg-lys-arg); PEPTIDE CK+ (ac-arg-1ys-arg-NH₂); and a copper derivative of his-gly-gly sold commercially as IAMIN, from Sigma (St. Louis, Mo.). Tetrapeptides and pentapeptides are also suitable for use herein. A preferred commercially available pentapeptide derivative composition is palmitoyl-1ys-thr-thr-1ys-ser (commercially available from Sederma France).

When included in the present compositions, peptides are preferably included in amounts of from about 1×10⁻⁶% to about 10%, more preferably from about 1×10⁻⁶% to about 0.1%, even more preferably from about 1×10⁻⁵% to about 0.01%, by weight of the composition. In certain compositions where the peptide is Camosine®, the compositions preferably contain from about 0.1% to about 5%, by weight of the composition, of such peptides. In other embodiments wherein the peptide or peptide-containing composition palmitoyl-lys-thr-thr-lys-ser and/or Biopeptide CL® are included, the compositions preferably contain from about 0.0001% to about 10%, of palmitoyl-lys-thr-thr-lys-ser and/or Biopeptide CL® peptide-containing composition.

Anti-inflammatory agents enhance the skin appearance benefits, by for example, contribution of uniformity and acceptable skin tone and/or color and are optionally included in the treatment composition of the present invention as well.

Preferably, the anti-inflammatory agent includes a steroidal anti-inflammatory agent and an non-steroidal anti-inflammatory agent. Preferred steroidal anti-inflammatory for use is hydrocortisone.

The variety of compounds encompassed by this group are well-known to those skilled in the art. For detailed disclosure of the chemical structure, synthesis, side effects, etc. of non-steroidal anti-inflammatory agents, reference may be had to standard texts, including Anti-inflammatory and Anti-Rheumatic Drugs, K. D. Rainsford, Vol. 1-III, CRC Press, Boca Raton, (1985), and Anti-inflammatory Agents, Chemistry and Pharmacology, 1, R. A. Scherrer, et al., Academic Press, New York (1974), each incorporated herein by reference.

So-called “natural” anti-inflammatory agents are also useful. Such agents may suitably be obtained as an extract by suitable physical and/or chemical isolation from natural sources (i.e., plants, fungi, by-products of microorganisms). For example, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, may be used.

Additional anti-inflammatory agents useful herein include compounds of the licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂₋₂₄ saturated or unsaturated esters of the acids, preferably C₁₀₋₂₄, more preferably C ₁₆₋₂₄.

The treatment composition of the present invention preferably contains a water soluble humectant as a skin benefit agent. Water soluble humectants are preferably included to provide moisturizing benefit to the skin. Further, water soluble humectants may help the dispersion of the water soluble thickening agents, and dissolving/dispersion of other components which are relatively difficult to process in an aqueous carrier.

Water soluble humectants useful herein include polyhydric alcohols such as glycerin, diglycerin, propylene glycol, dipropylene glycol, butylene glycol, hexylene glycol, sorbitol, ethoxylated glucose, 1,2-hexane diol, hexanetriol, erythritol, trehalose, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof.

Water soluble humectants useful herein also include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

The treatment composition of the present invention may include a chronic whitening agent as a skin care agent aw well. The chronic whitening agent useful herein refers to active ingredients that not only alter the appearance of the skin, but further improve hyperpigmentation as compared to pre-treatment. By definition, chronic is referred to continued topical application of the composition over an extended period during the subject's lifetime, preferably for a period of at least about one week, more preferably for a period of at least about one month, even more preferably for at least about three months, even more preferably for at least about one year. Typically, applications would be on the order of about once per day over such extended periods, while application rates can vary from about once per week up to about three times per day or more.

Useful chronic whitening agents useful herein include ascorbic acid compounds, vitamin B₃ compounds, azelaic acid, butyl hydroxy anisole, gallic acid and its derivatives, glycyrrhizinic acid, hydroquinoine, kojic acid, arbutin, mulberry extract, ergothioneine, and mixtures thereof. Among them, preferred are ascorbic acid compounds, vitamin B₃ compounds, and mixtures thereof. Use of combinations of chronic whitening agents are believed to be advantageous in that they may provide whitening benefit through different mechanisms.

The compositions of the present invention may include a skin tone changing agent as a skin benefit agent. The skin tone changing agent useful herein are selected from the group consisting of skin tone changing pigments, reflective particulate material, and mixtures thereof. Skin tone changing agents useful herein are those altering the appearance of the color and/or tone of the skin including, but not limited to, skin whitening. The skin tone changing agents have a particle size of, preferably at least about 100 nm. The skin tone changing pigments useful herein include, for example, talc, mica, silica, magnesium silicate, titanium oxide, zinc oxide, and titanium oxide coated mica.

The treatment compositions of the present invention are preferably in liquid form and include a water-soluble thickening polymer. The water soluble thickening polymers herein are water soluble or water miscible polymers, have the ability to increase the viscosity of the composition, and are compatible with other components used in the composition. The water-soluble thickening polymer is selected so that the liquid composition of the present composition has the desired viscosity of from about 500 mPa·s to about 60,000 mPa·s, preferably from about 1000 mPa·s to about 30,000 mPa·s, more preferably from about 2000 mPa·s to about 15,000 mPa·s.

Water soluble thickening polymers useful herein include anionic polymers and nonionic polymers. The water soluble thickening polymers useful herein include, for example, acrylic polymers, polyalkylene glycol polymers having a molecular weight of more than about 10000, celluloses and derivatives there of such as hydroxyethyl cellulose, polyvinylpyrrolidone, polyvinyl alcohol, gums such as guar gum and xanthan gum, carragenan, pectin, agar, quince seed (Cydonia oblonga Mill), starch (rice, corn, potato, wheat), algae colloids (algae extract), dextran, succinoglucan, pulleran, carboxymethyl starch, methylhydroxypropyl starch, sodium alginate, and alginic acid propylene glycol esters. Neutralizing agents may be included to neutralize the anionic thickening agents described hereinabove. Nonlimiting examples of such neutralizing agents include sodium hydroxide, potssium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

The treatment compositions of the present invention when in their preferred liquid form further include an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol. Preferably, the aqueous carrier is substantially water. Deionized water is preferably used. Water from natural sources including mineral cations can also be used, depending on the desired characteristic of the product.

The pH of the liquid compositions is preferably from about 4 to about 8. When skin benefit agents are included in the liquid composition, the pH may be adjusted to that which provides optimum efficacy of the active skin benefit agents. Buffers and other pH adjusting agents can be included to achieve the desirable pH. Suitable pH adjusters herein include acetates, phosphates, citrates, triethanolamines and carbonates.

In addition to the above described components, the composition of the present invention may further include preservatives and preservative enhancers such as water-soluble or dispersible preservatives including methyl paraben, propyl paraben, imidazolidinyl urea, Germall 115, methyl, ethyl, propyl and butyl esters of hydroxybenzoic acid, benzyl alcohol, EDTA, Bronopol (2-bromo-2-nitropropane-1,3-diol) and phenoxypropanol; ultraviolet light absorbers or scattering agents; sequestrants; anti-androgens; depilation agents; soluble or colloidally-soluble moisturizing agents such as hyaluronic acid and starch-grafted sodium polyacrylates such as SANWET® IM-1000, IM-1500 and IM-2500 available from Celanese Superabsorbent Materials, Portsmith, Va., USA and described in U.S. Pat. No. 4,076,663; proteins and polypeptides and derivatives thereof; organic hydroxy acids; drug astringents; external analgesics; film formers; anticaking agents; antifoaming agents; binders; coloring agents; perfumes, essential oils, and solubilizers thereof; natural extracts; guai-azulene; and yeast ferment filtrate.

The articles of the present invention are suitable for topical application on mammalian skin, in particular the human body, and even more particularly facial skin. The use of the article of the present invention can provide skin treatment benefits to the skin.

The article of the present invention is particularly advantageous in delivering the treatment composition and in preferred embodiments the skin care agents to the skin as the skin is exposed to an abundant amount of such agents over a lengthy period of time. Compared to when the composition is applied to the skin without the use of the insoluble substrate, the use of the article of the present invention, with the insoluble substrate as a delivery means over a lengthy period, is believed to provide better distribution and deposition of such agents, and better penetration of those agents which are percutaneously deliverable. Further, when an insoluble substrate having low air permeability is used, more effective penetration of the skin benefit agents into the skin is expected. The articles of the present invention may also provide emotional benefits to the user upon use, such as refreshing feel, and relaxation feel.

In one preferred embodiment, the article of the present invention and in particular the mask of the present invention is used to treat the human body by the steps of:
(a) providing an article having an elongation zone having at least a portion of the elongation zone being incrementally stretched, preferably the elongation zone comprises first and second regions as defined herein, and a treatment composition;
(b) bringing the article into contact with a portion of the human body;
(c) conforming the article to the contours of the body;
(d) allowing the article to contact the skin for a period of time effective to provide treatment of the skin
(e) and removing the article.

In preferred embodiments of the method, the article is allowed to stand on the skin for a period of time no longer than until any portion of the treatment composition is dried whereupon the article is removed from the skin. In the preferred embodiment of a facial mask, the mask is soaked or otherwise provided with the treatment composition and the mask is fitted to the facial skin by gently placing on the skin. The mask is then conformed to the skin for better fit and even distribution by pressing to the facial skin such as with one's finger tips.

By definition, “dried” refers to a state wherein water and other volatile components such as perfume, if included, evaporates from the water insoluble substrate, thereby leaving the substrate significantly less capable of delivering the treatment composition to the skin. Thus, once a portion of the mask is dried, even distribution of the composition cannot be expected. Further, when dried, the mask provides an unpleasant stiff and tough feeling to the skin when applied.

Because the composition of the present invention is easily dried via exposure to regular atmospheric conditions, articles which are provided wet are preferably housed in a hermetically sealed package during storage.

The period of time required until dried portions appear will depend on the atmosphere in which the use takes place, i.e. temperature, humidity, air circulation; and the structure and body temperature of the user. Typically, the composition should be designed so that no dried portions appear within a period of about 15 minutes when used in room temperature at a humidity of about 50%. When an insoluble substrate having low air permeability is used, the period of time by which the composition is dried can be prolonged, preferably from about 5 to about 45 minutes.

Accordingly, the present invention also relates to a method of treating the skin by use of the article of the present invention. The article of this embodiment is used by applying the article to the area of the skin to be treated such that the composition comes in contact with the skin. The remaining steps for using the article are the same as mentioned above. By using a preferred article comprising an occluded side, significant increase of penetration of the treatment composition may occur. Thus, application of such a treatment composition provides an effective method of delivering skin care actives to the facial skin. For effective delivery and distribution of the treatment composition, the article is applied to the skin for preferably a period of at least about 5 minutes, preferably from about 5 to about 45 minutes.

While the particular embodiments of the present invention have been illustrated and described, it would be readily apparent to those skilled in the art that various other changes and modifications can be made without departing from the spirit and scope of the present invention. It is therefore intended to cover in the appended claims all such changes and modifications that are within the scope of this invention.

### EXAMPLES

The following examples further describe and demonstrate embodiments within the scope of the present invention. The examples are given solely for the purposes of illustration and are not to be construed as limitations of the present invention, as many variations are possible without departing from the spirit and scope of the inventions.

### Examples 1-7

The articles of Examples 1-2, may be produced from about 2.5 g of a cotton substrate. The substrate is compressed between mating plates having opposed teeth and grooves to form an incremental stretch region in the area of the nose, cut and shaped according to FIG. 1. The substrate may then be immersed in a composition as set forth in Table 1 to load approximately 30 g of the compositions onto the substrate. The articles of Examples 3-5 may be produced as described with the substitution that the substrate material may be RFP-90 available from Daiwabo. Examples 6-7 may be produced as described with the substitution that the substrate material is DFS(SH)T-70 available from Daiwabo.

TABLE 1

| Components | Ex. 1 | Ex. 2 | Ex. 3 | Ex. 4 | Ex. 5 | Ex. 6 | Ex. 7 |
|---|---|---|---|---|---|---|---|
| Isopropyl isostearate | 0.5 | 0.5 | 0.5 | 0.5 | 0.5 | 0.5 | 0.5 |
| Sucrose polycottonseedate | 0.25 | — | 0.25 | — | 0.25 | — | 0.25 |
| Cetyl alcohol | 0.2 | 0.3 | 0.2 | 0.3 | 0.3 | 0.2 | 0.15 |
| Stearyl alcohol | 0.2 | 0.3 | 0.2 | 0.3 | 0.3 | 0.2 | 0.15 |
| Isohexadecane | — | 0.2 | — | — | — | — | — |
| Mineral oil | — | — | — | — | 2.0 | — | — |
| Coconut oil | — | 0.2 | — | — | 0.2 | — | — |
| PEG-100 stearate | 0.1 | 0.1 | 0.1 | 0.1 | 0.1 | 0.1 | 0.1 |
| Polysorbate 20 *1 | — | — | 0.3 | — | 0.3 | — | — |
| Steareth-21 | — | 0.2 | 0.1 | — | 0.5 | — | — |
| Cetearyl glucoside | 0.1 | — | 0.1 | 0.15 | 0.1 | 0.1 | — |
| Steareth-2 | — | 0.2 | — | — | 0.1 | — | — |
| Polyacrylamide *2 | 1.2 | — | 1.2 | — | 1.5 | — | 1.5 |
| Acrylates/C10-30 Alkyl Acrylate | — | 0.25 | — | 0.25 | — | — | — |
| Crosspolymer *3 | | | | | | | |
| Xanthan gum *4 | — | — | — | — | — | 0.5 | — |
| Glycerin | 3.0 | — | 3.0 | 3.0 | 3.0 | 3.0 | 3.0 |
| 1,3-butylene glycol | 5.0 | 5.0 | 5.0 | 5.0 | 5.0 | 5.0 | 5.0 |
| Dipropylene glycol | — | 3.0 | — | — | 4.0 | 4.0 | — |
| Titanium dioxide *5 | — | — | 0.1 | — | — | 0.1 | — |
| Ascorbyl glucoside *6 | — | 2.0 | — | — | — | 2.0 | — |
| Sodium Ascorbyl phosphate *7 | — | — | — | — | 0.5 | — | — |
| Niacinamide *8 | 3.5 | 2.0 | 2.0 | — | — | 1.0 | 4.0 |
| Panthenol *9 | — | — | 1.0 | — | — | — | 1.0 |
| Tocopherol Acetate *10 | — | 0.2 | — | — | — | — | — |
| Tocopherol Nicotinate *11 | 0.5 | — | — | 0.5 | 0.5 | 0.5 | — |
| Sodium salicylate | — | 0.2 | — | 0.5 | — | 0.5 | — |
| Mulberry extract *12 | 0.5 | — | 0.5 | — | 0.5 | 0.5 | — |
| Yeast Ferment Filtrate *13 | — | — | — | — | — | 30 | — |
| Natural extracts *14 | 0.05 | — | — | — | — | 0.01 | — |
| Ergothioneine *15 | — | — | 0.02 | — | — | 0.01 | — |
| Laminaria Digitata extract *16 | — | — | 0.1 | — | — | 0.01 | — |
| Cetearyl Glucoside and Cetearyl | — | — | — | — | — | — | 0.2 |
| Alcohol *17 | | | | | | | |
| Allantoin | — | — | — | — | — | — | 0.2 |
| Green Tea in Glycerin with Water *18 | — | — | — | — | — | — | 1.0 |
| Palmitoyl Lys Thr Thr Lys Ser *19 | — | — | — | — | — | — | 0.4 |
| Perfume | 0.05 | — | — | — | 0.1 | 0.03 | 0.02 |
| Benzyl alcohol | 0.15 | 0.15 | 0.15 | 0.15 | 0.15 | 0.15 | 0.25 |
| Ethyl paraben | 0.07 | 0.07 | 0.07 | 0.07 | 0.07 | 0.07 | 0.07 |
| Propyl paraben | 0.03 | 0.03 | 0.03 | 0.03 | 0.03 | 0.03 | 0.03 |
| Disodium EDTA | 0.1 | 0.1 | 0.05 | 0.1 | 0.1 | 0.1 | 0.1 |

| | | |
|---|---|---|
| NaOH | Adjust pH to 6-8 | — |
| Deionized Water | q.s. to 100% | |

Definitions of Components
*1 Polysorbate 20: Tween 20 available from ICI Surfactants
*2 Polyacrylamide: Sepigel 305 available from SEPPIC Inc.
*3 Acrylates/C10-30 Alkyl Acrylates Crosspolymer: Pemulen TR-2 available from B. F. Goodrich
*4 Xanthan gum: Keltrol available from Kelco
*5 Titanium dioxide: Kobo GLW75CAP-MP available from Kobo Products Inc.
*6 Ascorbyl glucoside: Available from Hayashibara.
*7 Sodium Ascorbyl phosphate: Stay-C available from Roche
*8 Niacinamide: Niacinamide available from Roche
*9 Panthenol: DL-Panthenol available from Roche
*10 Tocopherol acetate: Available from Eisai Co. Ltd.
*11 Tocopherol nicotinate: Available from Eisai Co. Ltd.
*12 Mulberry extract: mulberry extract BG available from Maruzen Pharmaceuticals
*13 Yeast Ferment Filtrate: SKII Pitera available from Kashiwayama
*14 Natural extracts: Phytelene EGX 243 [A mixture of Arnica Mountan Flower Extract, Hypericum Perforatum Extract, Ivy (Hedera Helix) extract, witchhazel (Hamamelis Virginiana) extract, grape (vitis vinifera) Leaf extract, and horse chestnut (aesculus hippocastanum) extract] available from Kaneda.
*15 Ergothioneine: Thiotaine available from AGI Dermatics
*16 Laminaria Digitata extract: Seanergilium BG available from Coletica
*17 Cetearyl Glucoside and Cetearyl Alcohol: Emulgade PL 68/50 from Cognis
*18 Green Tea in Glycerin with Water: available from Bio-Botanica, Hauppauge, NY
*19 Palmitoyl Lys Thr Thr Lys Ser: Peptide available from Sederma

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A treatment article comprising:
a) a water insoluble substrate comprising at least one elongation zone, at least a portion of said elongation zone being provided with incremental stretching; and
b) a treatment composition.

2. The treatment article of claim 1 wherein said article has at least a first region having a first surface pathlength and at least a second region having a second surface pathlength, said first region being incrementally stretched, said first and second surface pathlengths being measured generally parallel to a defined direction, said first surface pathlength being different than said second surface pathlength.

3. The treatment article of claim 2 wherein said first region and said second region are interconnected or interrelated to one another.

4. The treatment article of claim 2 wherein said article is provided with a plurality of first and second regions.

5. The treatment article of claim 2 wherein said first surface pathlength is greater than said second surface pathlength.

6. The treatment article of claim 5 wherein said second region is either an unaltered surface or an incrementally stretched surface.

7. The treatment article of claim 6 wherein said second region is an unaltered surface and said first surface pathlength is greater than said second surface pathlength by at least 3%.

8. The treatment article of claim 2 wherein said said first surface pathlength is greater than said second surface pathlength by at least 3%.

9. The treatment article of claim 1 wherein at least a portion of said water insoluble substrate comprises hydrophilic material.

10. The treatment article of claim 1 wherein said substrate is a non-woven substrate.

11. The treatment article of claim 10 wherein said non-woven substrate containing at least partially synthetic materials.

12. The treatment article of claim 10 wherein said nonwoven substrate is a three layer substrate.

13. The treatment article of claim 12 wherein said three layer substrate comprises an inner layer of a natural material and two outer layers of synthetic materials.

14. The treatment article of claim 1 wherein said water-insoluble substrate comprising a substrate having a skin facing side and an occluded side.

15. The treatment article of claim 14 wherein the occluded side has an air permeability of less than about 5 mg/cm²/min.

16. The treatment article of claim 14 wherein the occluded side of the substrate at least partially comprises a hydrophobic material.

17. The treatment article of claim 14 wherein the occluded side of said substrate is provided with a backsheet.

18. The treatment article of claim 14 wherein said skin facing side of the substrate at least partially comprises a hydrophilic material.

19. The treatment article of claim 1 wherein said water-insoluble substrate is configured to cover the majority of the area of the human face.

20. The treatment article of claim 1 wherein said water-insoluble substrate has a thickness of from about 100 μm to about 1 cm.

21. The treatment article of claim 1 wherein said article is a single piece facial mask.

22. The treatment article of claim 1 wherein said treatment composition includes at least one skin care active.

23. The treatment article of claim 22 wherein said skin care active is selected from the group consisting of skin texture improvement agents, anti-acne actives, anti-oxidants, peptides, anti-inflammatory agents, humectants, whitening agents, skin tone changing agents and combinations thereof.

24. The treatment article of claim 1 wherein said treatment composition is incorporated onto said water-insoluble substrate.

25. The treatment article of claim 1 wherein said water insoluble substrate has a machine direction and a cross direction, said first direction of incremental stretching being in the cross direction.

26. A cosmetic treatment method comprising the steps of providing a treatment article having a water insoluble substrate and a treatment composition, said substrate having at least one elongation zone, at least a portion of said elongation zone being provided with incremental stretching, applying said treatment article to a region of the body, conforming said article to the contours of the body, and allowing said article to contact the body for a period of time effective to provide cosmetic treatment.
